(19) United States
(12) Patent Application Publication
(10) Pub. No.: US 2023/0200846 A1
(43) Pub. Date: Jun. 29, 2023

(54) EXTENDABLE NEEDLE

(71) Applicants: THE JOHNS HOPKINS UNIVERSITY, Baltimore, MD (US); UNIVERSITY OF MARYLAND, BALTIMORE, Baltimore, MD (US)

(72) Inventors: Youseph Yazdi, Ellicott City, MD (US); Amanda Li, Cary, NC (US); Eshan Joshi, Richardson, TX (US); Diego Arevalo, Miami, FL (US); Asef Islam, Folsom, CA (US); Ernst Lippert Lozano, Blairstown, NJ (US); Chenyi Zhu, Baltimore, MD (US); Matthew Wang Zhao, Richardson, TX (US); Gabriel Fernandes, Miami, FL (US); Andrew Malinow, Baltimore, MD (US)

(21) Appl. No.: 17/797,970

(22) PCT Filed: Feb. 8, 2021
(86) PCT No.: PCT/US2021/017105

§ 371 Date: Aug. 5, 2022

Related U.S. Application Data
(60) Provisional application No. 62/971,411, filed on Feb. 7, 2020.

(51) Int. Cl. A61B 17/34

(52) U.S. Cl. CPC A61B 17/3401; A61B 17/3417; A61B 2017/3443; A61B 2017/346; A61B 2017/347

(57) ABSTRACT

The Tuohy needle of the present invention includes a hub design that allows for the needle to be extended mid-procedure from 3.5″ to 5″. The extendable needle of the present invention allows practitioners to better accommodate overweight and obese patients and introduce a simpler procedure workflow.

### CROSS REFERENCE TO RELATED APPLICATIONS

This application is a 35 U.S.C. § 371 U.S. national entry of International Application PCT/US2021/017105, having an international filing date of Feb. 8, 2021, which claims the benefit of U.S. Provisional Application No. 62/971,411, filed Feb. 7, 2020, the content of each of the aforementioned applications is herein incorporated by reference in their entirety.

### FIELD OF THE INVENTION

The present invention relates generally to medical devices. More particularly, the present invention relates to an extendable needle.

### BACKGROUND OF THE INVENTION

Over 11 million epidural anesthesia procedures are performed each year in the U.S. Most procedures begin with insertion of a standard 3.5″-long Tuohy (or Husted) needle. This length is often inadequate in reaching the epidural space of patients with a body mass index (BMI) of more than 30. Over the past few decades, obesity rates have been growing in the U.S. In the early 1990s, the rate was 23% for adults aged 20 and older; in 2016, the rate is 40%. Additionally, it is estimated that by 2030, roughly one-half of all men and women in the U.S will be obese. Unfortunately, when performing epidural anesthetics on obese or overweight patients, anesthesiologists face increased difficulty, failure rate, and time delays of the surgical procedure.

Anesthesiologists report that when attempting to insert a needle of inadequate length, it is routine (and often futile), to restart the procedure: choosing an anatomic space more rostral to the original, re-injecting intradermal local anesthesia, and reinitiating insertion of the needle. This makes the procedure more uncomfortable for the patient. At some hospitals, a longer needle may be available. However, substantial time is added to the procedure as the needle is retrieved and the procedure is reinitiated. This increases the cost of operating room time and risk of infection as the anesthesiologist removes sterile gloves, empties the sterile package containing the longer length needle onto the sterile field, and then immediately re-gloves. In addition, the sterile field is now open for an extended period of time, sometimes in an area considered non-sterile (e.g., labor room).

Finally, if longer needles are unavailable, the epidural technique is abandoned contributing to frustration for the anesthesiologist, anxiety and often unrelieved pain for the patient. As shown in FIG. 1, these factors contribute to creating a complicated workflow. FIG. 1 illustrates a flow diagram of a standard workflow for epidural anesthesia. The complicated standard workflow illustrated in FIG. 1 adds complexity and time to epidural anesthesia procedures for patients with long skin-to-epidural space depths that render 3.5″ Tuohy needles ineffective. Healthcare practitioners performing epidural anesthesia on obese patients need a way to consistently have the necessary needle length to reach the epidural space while maintaining the sterile field and maximizing patient comfort.

Therefore, it would be advantageous to provide a device for performing epidural anesthesia on patients with a long skin-to-epidural space distance.

### SUMMARY OF THE INVENTION

The foregoing needs are met, to a great extent, by the present invention, wherein in one aspect a device assembly includes a hub having inner and outer sheaths that are configured to telescope with respect to one another. The device includes a locking mechanism configured to lock the hub into an extended or retracted position. The device also includes a needle that is lengthened based on the hub being extended or retracted.

In accordance with an aspect of the present invention, the locking mechanism includes protrusions for engaging the hub. The inner sheath includes indentations for coupling with the locking mechanism. The locking mechanism is rotated about the inner sheath in order to further engage the indentations. The outer sheath comprises wings.

In accordance with another aspect of the present invention, a device assembly includes a hub having inner and outer sheaths that are configured to telescope with respect to one another. The device also includes a locking mechanism configured to lock the inner and outer sheaths of the hub into a predetermined configuration. Additionally, the device includes a needle that is lengthened based on a position of the hub as extended or retracted.

In accordance with still another aspect of the present invention, the locking mechanism includes an internal protrusion for engaging the hub. The inner sheath includes indentations for coupling with the locking mechanism. The locking mechanism is rotated about the inner sheath in order to further engage the indentations. The outer sheath includes wings. The wings are configured to move towards the physician, when the needle is extended. The inner and outer sheaths lock in the retracted position with rotational movement with respect to one another. A rotational structure of the device is limited to the hub such that rotational locking and unlocking of the inner and outer sheaths does not rotate a shaft of the needle. The needle is smooth along its entire surface whether in the non-extended or extended position. The inner and outer sheaths telescope slidably with respect to one another. The locking mechanism includes an outer tab for removal. The locking mechanism is configured to lock the inner and outer sheath together in a retracted position. The locking mechanism is configured to lock the inner and outer sheath together in an extended position.

In accordance with yet another aspect of the present invention, a device assembly includes a hub having inner and outer sheaths that are configured to telescope with respect to one another. The inner and outer sheaths are further configured to lock in an extended or retracted position. The device includes needle that is lengthened based on a position of the hub as extended or retracted.

In accordance with still another aspect of the present invention, the outer sheath includes wings. The wings are configured to move towards the physician, when the needle is extended. The inner and outer sheaths lock in the retracted position with rotational movement with respect to one another. A rotational structure of the device is limited to the hub such that rotational locking and unlocking of the inner and outer sheaths does not rotate a shaft of the needle. The needle is smooth along its entire surface whether in the non-extended or extended position.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a flow diagram of a standard workflow for epidural anesthesia.

FIGS. 2A-2C illustrate views of an expandable epidural needle, according to an embodiment of the present invention.

FIGS. 3A-3D illustrate views of a hub for an expandable epidural needle, according to an embodiment of the present invention.

FIGS. 4A-4D illustrate exploded views of a hub component for an expandable epidural needle, according to an embodiment of the present invention.

FIGS. 5A-5D illustrate views of an outer hub component for an expandable epidural needle, according to an embodiment of the present invention.

FIGS. 6A-6D illustrate views of an inner hub component for an expandable epidural needle, according to an embodiment of the present invention.

FIGS. 7A-7D illustrate views of a locking knob component for an expandable epidural needle, according to an embodiment of the present invention.

FIG. 8 illustrates a simplified workflow that results from using the device associated with the present invention.

FIG. 9 illustrates force data for the locking mechanism, according to an embodiment of the present invention.

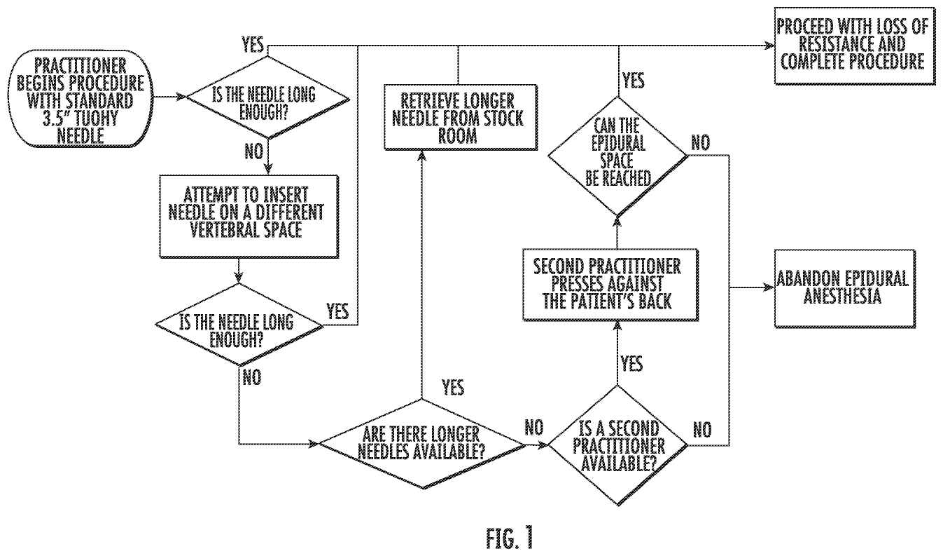

### DETAILED DESCRIPTION

The presently disclosed subject matter now will be described more fully hereinafter with reference to the accompanying Drawings, in which some but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The Tuohy needle of the present invention includes a hub design that allows for the needle to be extended mid-procedure from 3.5″ to 5″. The extendable needle of the present invention allows practitioners to better accommodate overweight and obese patients and introduce a simpler procedure workflow.

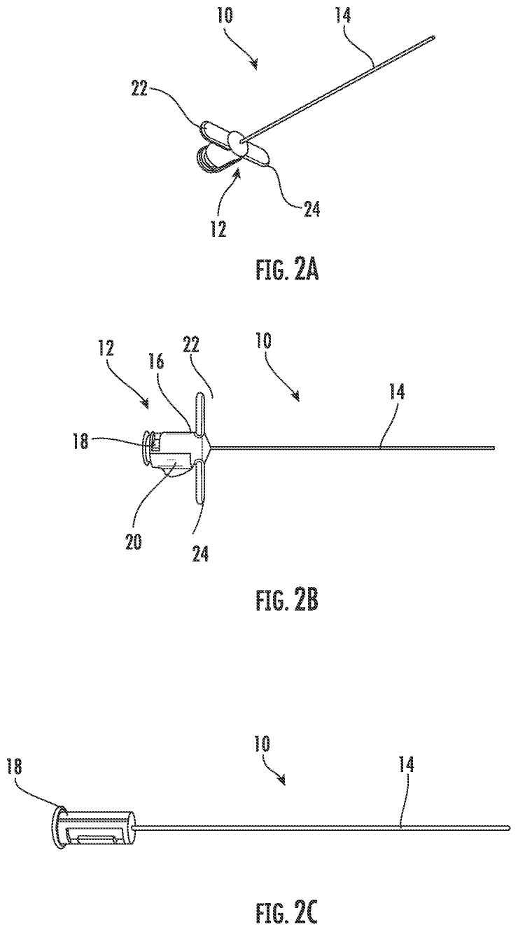

FIGS. 2A-2C illustrate views of an expandable epidural needle, according to an embodiment of the present invention. As illustrated in FIGS. 2A-2C the expandable epidural device 10 includes a hub 12 and a needle 14. The hub 12 is configured with an outer hub component 16, an inner hub component 18, and a locking mechanism 20. The inner hub component 16 and the outer hub component 18 are configured to telescope relative to one another, in order to shorten the length of the hub 12 and extend the length of the needle 14. A locking mechanism 20 is positioned on the hub 12 and prevents transverse movement between the outer hub component 16 and the inner hub component 18. Removal of the locking mechanism 20 allows the outer hub component 16 to be retracted in order to expose extra length of the needle 14, when required. The outer hub component 16 has indentations, described in further detail below, which fit with the protrusions of the locking ring 20 to impede transverse movement between the two components 16, 18. Additionally, because the two components 16, 18 are situated only at the hub 12, rotational locking/unlocking of the two components 16, 18 during needle extension/retraction avoids rotating the shaft of the needle 14. The hub 12 was designed such that extending the needle 14 involves movement of wings 22, 24 towards the anesthesiologist, decreasing the possibility that a tip of the needle 14 unintentionally advances further into the patient's back.

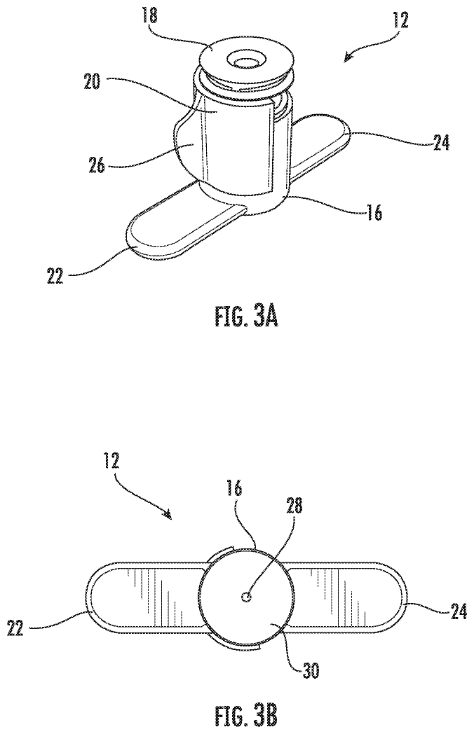

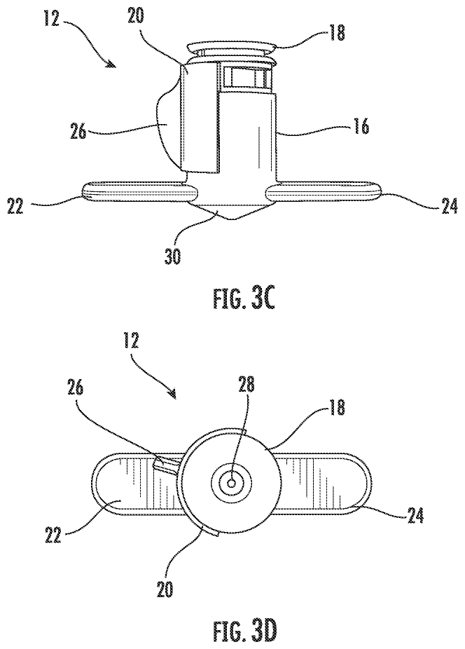

FIGS. 3A-3D illustrate views of a hub for an expandable epidural needle, according to an embodiment of the present invention. As described with respect to FIGS. 2A-2C, the hub 12 includes the outer hub component 16, the inner hub component 18, and the locking mechanism 20. The outer hub component 16 and the inner hub component 18 telescope relative to one another, in order to shorten the length of the hub 12 and extend the length of 5 the needle 14. The locking mechanism 20 is positioned on the hub 12 and prevents transverse movement between the outer hub component 16 and the inner hub component 18. Removal of the locking mechanism 20 allows the outer hub component 16 to be retracted in order to expose extra length of the needle 14, when required. The locking mechanism 20 includes a tab 26 to ease removal of the locking mechanism 20 from the hub. After the locking mechanism is removed, wings 22, 24 are moved towards the anesthesiologist to shorten the hub 12 and lengthen the needle 14. While a locking configuration with protrusions and indentations is described herein, any suitable means of locking the inner and outer hub components could be used, as is known to or conceivable to one of the skills in the art. The needle 14 is disposed in opening 28, defined by a base 30 of the hub 12. It should be noted that the locking mechanism 20 can be integrated within the inner and outer hub components 16, 18. Alternately the locking mechanism 20 can be a separable and removable component that is not molded with the inner and outer hub components 16, 18.

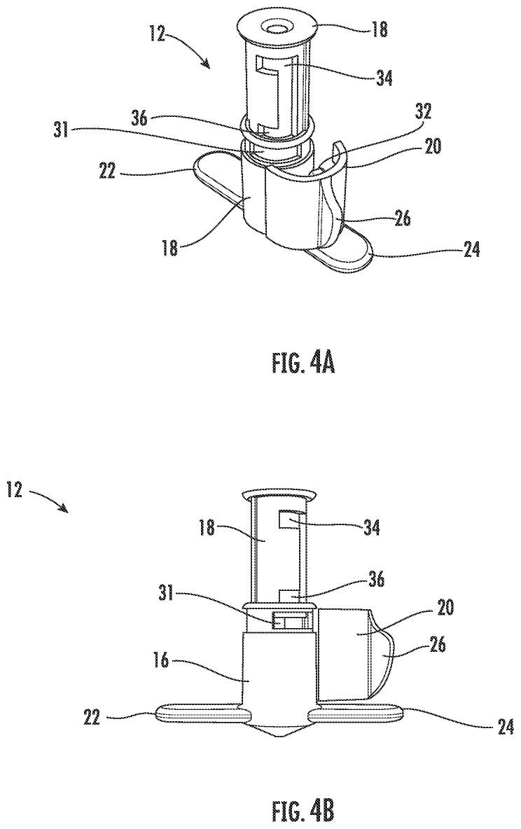

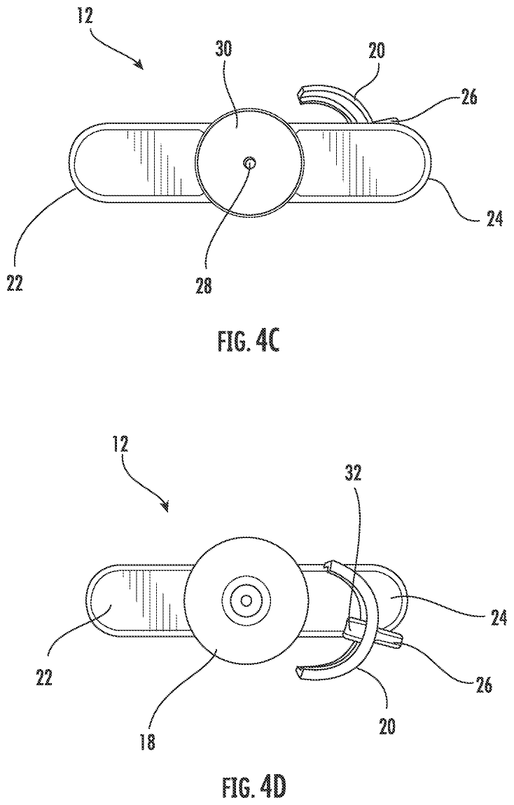

FIGS. 4A-4D illustrate exploded views of a hub component for an expandable epidural needle, according to an embodiment of the present invention. The locking mechanism 20 is positioned on the hub 12. The locking mechanism 20 includes protrusion 32 that fits within indentations 34, 36 on the inner hub component 18. The indentations 34, 36 allow for the protrusion 32 to be rotated partially around the inner hub component 18, in order to couple the locking mechanism 20 to the inner hub component 18. Alternately, the indentations for coupling the locking mechanism to the hub 12 can be positioned on the outer hub component 16. The outer hub can include openings 31 to allow the protrusion 32 of the locking mechanism to engage the indentations 34 36 of the inner hub component 18. Removal of the locking mechanism 20 allows the outer hub component 16 to be retracted in order to expose extra length of the needle 12, when required. In order to remove the locking mechanism 20, the locking mechanism 20 is rotated and removed. The locking mechanism 20 includes a tab 26 to ease removal of the locking mechanism 20 from the hub. After the locking mechanism is removed, wings 22, 24 are moved towards the anesthesiologist to shorten the hub 12 and lengthen the needle 14. The needle 14 is disposed in opening 28, defined by a base 30 of the hub 12. In some embodiments the outer hub component 16 can then be locked to the inner hub component 14 in order to prevent movement of the components of the hub 12 during the procedure. Locking the outer hub 16 to the inner hub 18 can be achieved through rotation and engagement of protrusions and indentations, frictional fit, or any other suitable mechanism known to or conceivable to one of skill in the art. In other embodiments, the locking mechanism 20 can be replaced after the shortening of the hub 12 to lock the inner and outer hub components 16, 18 in the compressed position.

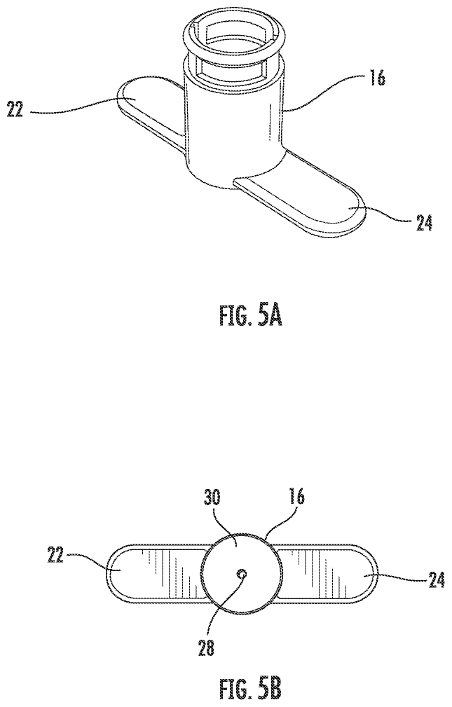

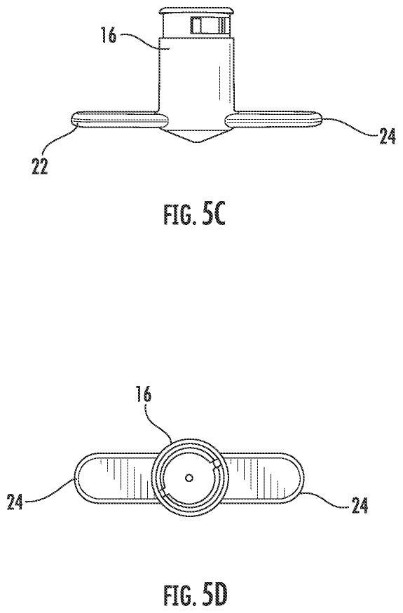

FIGS. 5A-5D illustrate views of an outer hub component for an expandable epidural needle, according to an embodiment of the present invention. The outer hub component 16 includes wings 22 and 24. The outer hub component 16 also defines an opening 38 into which the inner hub component (not pictured) is disposed. The needle 14 is dis-posed in opening 28, defined by a base 30 of the hub 12.

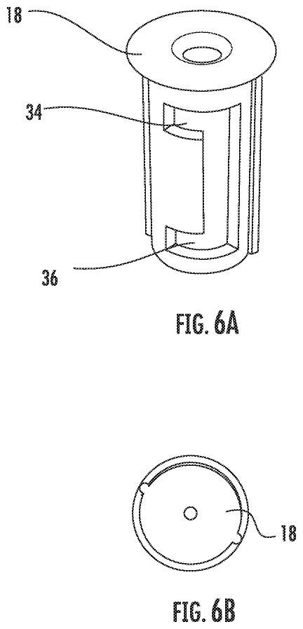

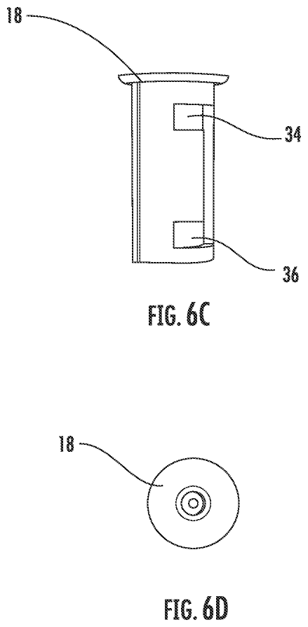

FIGS. 6A-6D illustrate views of an inner hub component for an expandable epidural needle, according to an embodiment of the present invention. The inner hub component 18 is configured to be disposed within the outer hub component (not pictured). The indentations 34 allow for the protrusion of the locking mechanism (not pictured) to be rotated partially around the inner hub component 18, in order to couple the locking mechanism to the inner hub component 18. The locking mechanism (not pictured) can be engaged into either indentation 34, 36 depending on the length of needle necessary for the epidural procedure.

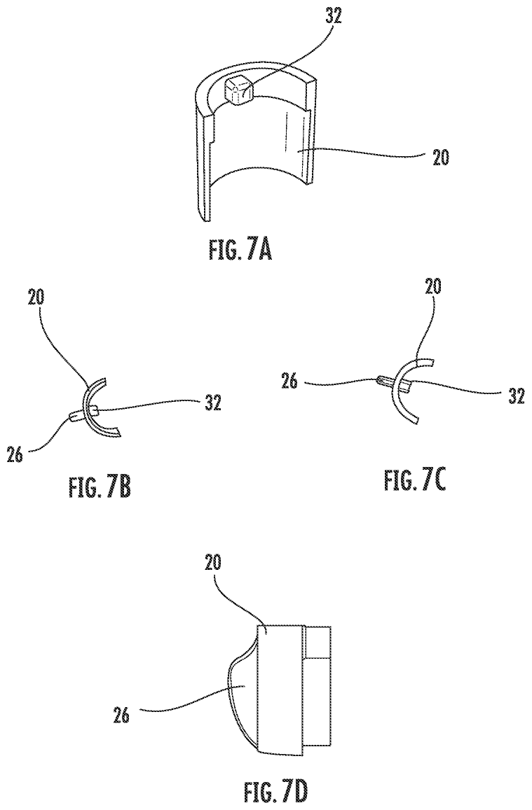

FIGS. 7A-7D illustrate views of a locking mechanism for an expandable epidural needle according to an embodiment of the present invention. The locking mechanism 20 includes protrusion 32 that fits within indentations on the inner hub component (not pictured). The locking mechanism also includes tab 26 to make removal of the locking mechanism 20 easier for the physician.

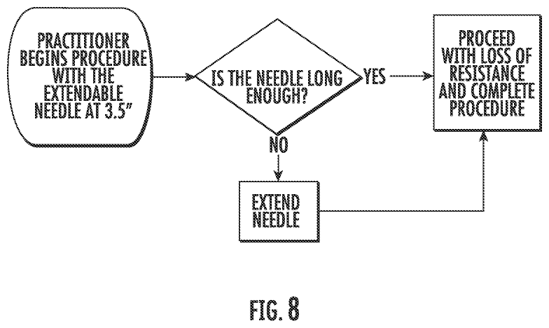

FIG. 8 illustrates a simplified workflow that results from using the device associated with the present invention. In the new workflow, associated with the present invention, the practitioner begins with the extendable needle at the traditional 3.5 inch length. The practitioner determines whether the needle is long enough. If yes, the practitioner will proceed with loss of resistance and complete the epidural procedure. If not, the practitioner will extend the needle and then proceed with loss of resistance and complete the epidural procedure.

Patient and physician needs and market trends were investigated through expert interviews and physician shadowing. Based on numerical data regarding the barriers to market entry, observational data from shadowing sessions, and physician interviews, the following design requirements were established for a new needle design:
The needle must be compatible with ranges of obese anatomy (BMI≥30), which indicates that it must be able to have extensions to traverse the various epidural depths (7.5-12.2 cm or 3-5 in.)
   The three classes of obesity are associated with different depths from the skin to the epidural space. Normal BMI patients have an average epidural space of 7.5 cm or 2.95 inches, class I obesity patients (30≤BMI≤35) have a depth of 10 cm or 3.94 inches, class II (35≤BMI≤40) have a depth of 10.4 cm or 4.09 inches, and class III (40≤BMI≤66.8) have a depth of 12.5 cm or 4.92 inches. To accommodate all obese patients, the needle length must be able to traverse epidural depths ranging from normal (3 inches) to class III obese (5 inches).
For all obese anatomy, less than 1 inch (2.5 cm) of the needle, measured from the wings distally from the patient's skin to the physician, should be outside the patient's back
   A clinician-verified statement supports the notion that leaving extraneous needle length outside the patient increases both practitioner discomfort and risk of unintentionally jarring the needle and perhaps damaging tissue.
Needle must withstand extreme axial force of 17 N
   The extension points on the needle may be weak points that need to withstand the maximum force experienced in epidural anesthesia. The greatest force that the needle experiences when it hits bone has been measured to be approximately 17 N. At the same time, the needle must conform to ISO standards for stiffness and resistance to breakage. This is vital to ensure the needle is able to penetrate tissue without buckling.
All parts of the needle should be pressure sealed to facilitate the loss of resistance technique.
   The current loss of resistance technique relies on the needle and syringe being completely sealed so that the air or saline in the syringe is only able to be ejected easily once the epidural space is reached.
Interface with currently existing syringes and catheters—have a Luer lock/slip conforming to properties and dimensions of ISO 80369-6:2016
   The device must be compatible with the current standard of care, which involves connecting to the standard loss of resistance syringes.
Time added to the procedure when using the device cannot be more than 5 minutes.
   From shadowing experience and expert feedback, the average epidural procedure is around 30 minutes total. The time associated currently to retrieve a new needle and restart a procedure is around 5-10 minutes. The solution should be a faster alternative to the current method.
Depth marks at every 1 cm on the needle to allow practitioners to know how deep in the patient the needle is.
   Visual markers are important for physicians to have a mental representation of how deep the needle is and know an approximate depth of the epidural space. Depth marks allow the anesthesiologist to gauge if a previously threaded catheter has unintentionally been withdrawn (during initial fixation at the skin and sterile dressing or with a change in dressing). Burnishing needles with depth marks is a feature implemented in most currently available epidural needles.
Cannot increase cost of kit by 100%
   Epidural market analysts confirmed that doubling the cost of epidural kits that have some new advantage would not hinder selling potential. However, severe cost escalation could hinder adoption of the device.

The need for a longer needle for obese patients, who have a larger corresponding skin-to-epidural depth, is clearly demonstrated since the 3.5″ standard is not long enough to traverse the longer depths associated with BMI≥30. However, starting the procedure with the longest epidural needle is also not recommended since longer needles are harder to manipulate and are more difficult to control. During clinician interviews, it was emphasized that the extraneous length of the needle outside of the patient's back can also pose the risk of having healthcare practitioners unintentionally jar the needle. Therefore, patients need an ideal epidural needle length that is not too long and not too short.

Two aspects of the design were tested. First, the integrity of the locking mechanism was tested in enduring extreme axial force encountered during an epidural anesthesia procedure. Second, design usability was tested by conducting a usability study that provides physician feedback on the prototype. The axial force test was completed using Finite Element Analysis. A safety factor of 2 was assumed to ensure that a real design would not fail unless the actual stress was half the lowest stress resulting in material failure (as Safety Factor=Material Strength/Actual Stress). To maintain needle durability during maximum stress of the procedure (17 N), the material strength, or maximum allowable stress, was chosen to be twice this value. A Margin of Safety=Safety Factor −1 correction factor was employed, yielding a Safety Factor threshold of 3. This threshold allows twice the applied load (perpendicular to hub) to be added to the applied load before failure begins to occur.

During the usability study, physicians were asked to use the prototyped needle hub, in conjunction with a Tuohy needle, on a spine model. Physicians will follow a procedure that guides them through the extension of the device. Physicians will be asked to comment on the intuitiveness of the procedure and device. After completing the procedure on the spine model (which serves as a makeshift obese patient), physicians fill out a questionnaire to provide input on usability of the device and whether the solution concept is compatible with select design requirements.

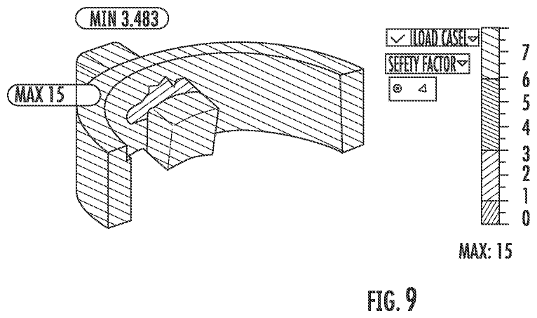

First, the locking mechanism was tested, using polypropylene parameters, to withstand 17 N during finite element analysis through Fusion 360. FIG. 9 illustrates force data for the locking mechanism, according to an embodiment of the present invention. As shown in FIG. 9, the device has an actual minimum safety factor of 3.483, which indicates that the locking mechanism is sufficient to withstand all forces experienced in epidural anesthesia as it is exceeds 3, which is the threshold needed to remain within the margin of safety. Next, a questionnaire usability study was conducted. The analytic sample for the questionnaire study was five faculty anesthesiologists, selected randomly from a group of attending anesthesiologists at the University of Maryland Medical Center. Participants covered a wide spread of number of epidural anesthetics performed; prior to the study, one physician had performed 0-50 procedures, two physicians had performed 100-500 procedures, and the other two physicians had performed more than 500 procedures.

Anesthesiologists were asked to use the extendable epidural needle on a spine model and to critique various aspects of its usability. In the questionnaire, physicians are inquired about the ease-of-use of the device, how it would change the current workflow, and how it would affect risk of contamination, time of procedure, and patient comfort. The anesthesiologists were given a 3D-printed (VeroClear and Rigur) prototype of the extendable needle. They were instructed to perform a mock epidural anesthetic on a spine model: insert the epidural needle, extend the needle, thread a catheter, and withdraw the needle over the catheter.

Table 1 lists questions and physician responses. Results demonstrate that there would be either no change or a slight decrease in procedure difficulty and risk of infection. Additionally, anesthesiologists believed that the use of the device would lead to a decrease in procedure time and increase in patient comfort. Suggestions for improvement included introducing an auto-locking mechanism to avoid needle instability while re-locking the needle after its extension, incorporating a transparent Tuohy needle hub, and interchanging the locking and unlocking directions. Overall, the results from this survey indicate that an extendable Tuohy needle might improve patient and physician experience during an epidural anesthesia procedure performed on obese patients.

Based on the results of the usability survey, the concept and design seem to be favorable for anesthesiologists. The design of an extendable needle potentially has applications to other anesthetic procedures (such as peripheral nerve blocks) and to other fields of medicine, in which extendable needles/tubing would be useful for aspiration of tissue/fluid (e.g. biopsy), injection of a drug or threading of a catheter.

Responses to the questionnaire indicate that the prototype design did not threaten to increase risks of contamination or difficulty of overall procedure, and could potentially reduce procedure time and increase patient comfort/decrease patient discomfort. Nonetheless, based on the results of questionnaire and physician feedback, in future design revisions, several considerations could be made. As all anesthesiologists answered that the design neither complicates nor eases the procedure, the device and procedure should be simplified. To this end, an auto-locking mechanism could be developed, allowing the ring mechanism to “self-lock” after the outer sheath is retracted. Incorporation of a transparent hub would allow physicians to see standard one-centimeter markings on the catheter during catheter threading and withdrawal of the needle over the catheter. Interchanging the design's locking and unlocking directions, thus making a counterclockwise turn an unlocking motion and a clockwise turn a locking motion, could also increase the design's intuitiveness.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

TABLE 1

| | Figures displaying number of |
|---|---|
| | physicians that provided a particular |
| Questions | answer choice for a given question |
| 1. Does the added functionality change | 60% of the sample group said that the |
| the difficulty of advancing the needle into | design would not increase difficulty of |
| the epidural space? | advancing needle into epidural space |
| [1]: Hub design very seriously increases | |
| difficulty | |
| [2]: Hub design slightly or moderately | |
| increases difficulty | |
| [3]: Hub design does not increase | |
| difficulty | |
| [4]: Hub design slightly or moderately | |
| decreases difficulty | |
| [5]: Hub design greatly reduces difficulty | |
| 2. Do you think that the difficulty of | 80% of the sample group was neutral as |
| threading the catheter will change with | to whether the design would change |
| this design? | difficulty of threading the catheter |
| [1]: Hub design very seriously increases | |
| difficulty | |
| [2]: Hub design slightly or moderately | |
| increases difficulty | |
| [3]: Hub design does not increase | |
| difficulty | |
| [4]: Hub design slightly or moderately | |
| decreases difficulty | |
| [5]: Hub design greatly reduces difficulty | |
| 3. After insertion of the desired length of | 100% of the sample group was neutral |
| catheter, will the current design change | as to whether the design would change |
| the difficulty of withdrawing the needle | difficulty of withdrawing the needle |
| over the catheter? | over the catheter |
| [1]: Hub design very seriously increases | |
| difficulty | |
| [2]: Hub design slightly or moderately | |
| increases difficulty | |
| [3]: Hub design does not increase | |
| difficulty | |
| [4]: Hub design slightly or moderately | |
| decreases difficulty | |
| [51: Hub design greatly reduces difficulty | |
| 4. Either prior to or during attempted | The needle hub design does not alter |
| insertion of an epidural needle in morbid | physician confidence, and for some |
| and supermorbid obese patients, how | physicians improved confidence, in |
| confident are you that this needle hub | being able to use the appropriate needle |
| design will allow you to select the ideal | length for obese patients |
| needle length for the patient? | |
| [1] Needle design degrades confidence | |
| [2] Needle design does not change | |
| confidence; provides confirmation of my | |
| original decision | |
| [3] Needle design improves confidence | |
| by providing reassurance of my original | |
| decision | |
| [4] Needle design improves confidence | |
| by providing additional information to | |
| reach my decision | |
| [5] It would have been extremely | |
| challenging without this needle design | |
| 5. Compared to current practice of | 100% said the design would either |
| switching from a 3.5″ epidural needle to | decrease or not changer risk of |
| available longer (5″-7″) epidural needles, | contamination of the sterile field |
| does a kit (or minikit) with an extendable | |
| needle change risk of contamination of | |
| the sterile field? | |
| [1]: Great increase in risk of | |
| contamination | |
| [2]: Slight to moderate increase in risk of | |
| contamination | |
| [3]: No significant change in risk of | |
| contamination | |
| [4]: Slight to moderate decrease in risk of | |
| contamination | |
| [5]: Great decrease in risk of | |
| contamination | |
| 6. Do you routinely stock longer (5″-7″) | 3 physicians answered Yes, while 2 |
| epidural needles on your epidural carts in | physicians answered No. This is a |
| all anesthetizing locations (e.g., L&D, | small sample size, but here 40% of the |
| General OR, Acute or Regional Pain | questioned physicians indicated that |
| Service)? | obtaining longer needles requires |
| [1]: Yes | leaving the anesthetizing location |
| [2]: No | |
| 7. Accounting for both the time needed | 100% said the design would save time. |
| for (multiple?) failed attempts to insert a | This makes sense because the design is |
| 3.5″ epidural needle in obese, morbid, | intended to change the need for |
| and supermorbid obese patients and time | enlisting someone to retrieve a longer |
| spent procuring and adding the | epidural needle or calling for the |
| extendable needle to the sterile field, | assistance of another practitioner in |
| please estimate the additional time | completing the epidural procedure. |
| saved/needed using this extendable | |
| Tuohy needle as compared to your | |
| current practice during epidural | |
| anesthetics for morbid and super-morbid | |
| obese patients? | |
| [1]: Adds extra time | |
| [2]: Saves 0-3 minutes | |
| [3]: Saves 3-5 minutes | |
| [4]: Saves 5-10 minutes | |
| [5]: Saves >10 minutes | |
| 8. Does the use of an extendable needle | 100% said the design would increase |
| change patient comfort during the | patient comfort. This makes sense |
| procedure compared to multiple attempts | because the design is intended to |
| with epidural needles routinely stocked | decrease patient discomfort by |
| on your cart before switching to longer | preventing multiple passes with |
| needles? | the standard Tuohy needle and a longer |
| [1]: Large increase in patient discomfort | Tuohy needle |
| [2]: Slight or moderate increase in patient | |
| discomfort | |
| [3]: No change in comfort levels | |
| [4]: Slight or moderate decrease in | |
| patient discomfort | |
| [5]: Large decrease in patient discomfort | |
| 9. Do you believe that immediate | All physicians answered Yes. Indeed, |
| availability of a (mini-) kit including an | the extending ability of the new needle |
| extendable needle will change the need | hub design should mitigate the need of |
| for enlisting someone to retrieve a longer | switching to a longer needle and avoid |
| epidural needle or calling for the | the second practitioner needed to press |
| assistance of another practitioner in | on patient's back during epidural |
| completing the epidural procedure? | anesthesia procedures on obese patients |
| [1] Yes | |
| [2] No | |
| 10. Compared to the current utilization of | The questioned physicians believed |
| epidural needles of different lengths, an | that the design would either maintain or |
| anesthesiologist/anesthetist using an | decrease difficulty of epidural |
| extendable needle will find induction of | anesthesia procedures on obese |
| performing an epidural anesthesia in | patients. Physicians spoke to |
| obese, morbid-, and super-morbid obese | the device's ability to save time in |
| patients: | future procedures on obese patients |
| [1]: Largely more difficult | (avoid restarting procedure with longer |
| [2]: Slightly to moderately more difficult | needle), and decrease in physician |
| [3]: No change in difficulty | frustration due to decreased need to |
| [4]: Slightly to moderately easier | look for longer needle and restart |
| [5f Significantly easier | procedure |
| 11. How many epidural anesthetics have | No correlations were found between |
| you performed? | number of procedures performed and |
| [1]: 0-50 | responses to other questions, but the |
| [2]: 50-100 | sample of physicians did indeed have a |
| [3]: 100-500 | fair spread of number of epidural |
| [4]: 500-1000 | anesthetics performed |
| [5]: More than 1000 | |

What is claimed is:

1. A device assembly comprising:
a hub having inner and outer sheaths that are configured to telescope with respect to one another;
a locking mechanism configured to lock the inner and outer sheaths of the hub into a predetermined configuration; and
a needle that is lengthened based on a position of the hub as extended or retracted.

2. The device of claim 1 wherein the locking mechanism comprises an internal protrusion for engaging the hub.

3. The device of claim 2 wherein the inner sheath comprises indentations for coupling with the locking mechanism.

4. The device of claim 3 wherein the locking mechanism is rotated about the inner sheath in order to further engage the indentations.

5. The device of claim 1 wherein the outer sheath comprises wings.

6. The device of claim 5 wherein the wings are configured to move towards the physician, when the needle is extended.

7. The device of claim 1 wherein the inner and outer sheaths lock in the retracted position with rotational movement with respect to one another.

8. The device of claim 7 wherein a rotational structure of the device is limited to the hub such that rotational locking and unlocking of the inner and outer sheaths does not rotate a shaft of the needle.

9. The device of claim 1 wherein the needle is smooth along its entire surface whether in the non-extended or extended position.

10. The device of claim 1 where the inner and outer sheaths telescope slidably with respect to one another.

11. The device of claim 1 wherein the locking mechanism comprises an outer tab for removal.

12. The device of claim 1 wherein the locking mechanism is configured to lock the inner and outer sheath together in a retracted position.

13. The device of claim 1 wherein the locking mechanism is configured to lock the inner and outer sheath together in an extended position.

14. A device assembly comprising:
a hub having inner and outer sheaths that are configured to telescope with respect to one another, wherein the inner and outer sheaths are further configured to lock in an extended or retracted position; and
a needle that is lengthened based on a position of the hub as extended or retracted.

15. The device of claim 14 wherein the outer sheath comprises wings.

16. The device of claim 15 wherein the wings are configured to move towards the physician, when the needle is extended.

17. The device of claim 14 wherein the inner and outer sheaths lock in the retracted position with rotational movement with respect to one another.

18. The device of claim 17 wherein a rotational structure of the device is limited to the hub such that rotational locking and unlocking of the inner and outer sheaths does not rotate a shaft of the needle.

19. The device of claim 14 wherein the needle is smooth along its entire surface whether in the non-extended or extended position.

20. The device of claim 14 further comprising a locking mechanism configured to hold the inner and outer sheaths in a retracted or extended position.
